(19) United States
(12) Patent Application Publication
(10) Pub. No.: US 2003/0091981 A1
(43) Pub. Date: May 15, 2003

(54) Funtional assay for genes involved in xylogenesis

(75) Inventors: Sathish Puthigae, Auckland (NZ); Jonathan Phillips, Remnera (NZ); Barry Flinn, Frederiction (CA); James G. Murison, Sandringham (NZ)

Correspondence Address: SPECKMAN LAW GROUP, 1501 WESTERN AVE, SUITE 100, SEATTLE, WA, 98101, US

(73) Assignee: GENESIS RESEARCH AND DEVELOPMENT CORP. LTD., Parnell (NZ)

(21) Appl. No.: 10/291,447

(22) Filed: Nov. 8, 2002

Related U.S. Application Data
(60) Provisional application No. 60/345,397, filed on Nov. 9, 2001.

(51) Int. Cl. C12Q 1/00; C12Q 1/68; A01H 1/00; C12N 15/82

(52) U.S. Cl. 435/4; 435/6; 800/284

(57) ABSTRACT

The present application relates to a functional assay for high-throughput screening and identification of plant genes involved in xylogenesis and secondary cell wall formation. The assay involves transforming Zinnia mesophyll cells in vitro under non-inducing or transdifferentiation-inducing conditions with DNA constructs comprising one or more polynucleotide sequences toa be tested and comparing the expression of the sequences in the non-induced and transdifferentiating cells. The assay is particularly useful for identifying promoters that are active in xylem and xylem-forming tissues and their transcriptional regulators.

### CROSS-REFERENCE TO RELATED APPLICATIONS

This application claims benefit of priority to U.S. Provisional Patent Application No. 60/345,397, filed Nov. 9, 2001.

### FIELD OF THE INVENTION

This application relates to a functional assay for identifying genes that are active in xylogenesis and their transcriptional regulators. The assay involves the transient expression of transgenes in Zinnia elegans mesophyll cells, which are induced to differentiate into tracheary elements.

### BACKGROUND OF THE INVENTION

An understanding of the molecular basis of wood formation and the identification of tree genes involved in xylogenesis is of considerable importance for improving wood properties in commercial tree species. Wood fibers are composed of cellulose and hemicellulose upon which lignin and structural proteins are deposited. The physico-chemical properties of the wood are determined by the arrangement and composition of these components, which is controlled by genes that are expressed during xylogenesis. Xylem is a water- and solute-conducting vessel of higher plants which forms from the end-to-end association of tracheary elements, the terminally differentiated acellular end products of xylogenesis. Tracheary elements develop from cambial cells in the vascular system. These cells elongate, deposit a patterned lignified secondary cell wall, and undergo programmed cell death. Cell autolysis removes the cellular contents and the residual hollow conducting cell is the tracheary element (TE). The secondary cell walls of the tracheary elements contribute to the mechanical support which the xylem provides to the plant. Secondary xylem (wood) develops from the vascular cambium and is used commercially for the production of lumber, paper and fiber.

Various approaches have been used to identify genes that affect xylem formation in trees. For example, large scale cambium/xylem EST libraries can be prepared from a tree species of interest. Comparison of library EST sequences with sequences of other plant species having known functions related to xylogenesis (e.g., cell wall proteins, lignin biosynthetic enzymes, enzymes of carbohydrate metabolism, and some transcriptional regulators) is used to assign putative functions to the ESTs in the tree species of interest. Expression analysis can be used to identify genes that are differentially expressed in different types of wood in an individual tree and the expression of particular genes or sets of genes is correlated with wood properties.

Plant model systems, such as Arabidopsis, can be used to generate and characterize mutations that affect cell wall synthesis and xylem formation. The affected genes are cloned and sequenced and used to identify tree genes that are structural homologs.

Needless to say, these approaches are time consuming and laborious, and are limited to identifying tree genes whose functions are already known in other species and whose sequences are compiled in accessible databases.

An alternative approach is to study xylogenesis in plant model systems that are induced to regenerate xylem or to differentiate into xylem in vitro (reviewed in V. Raghavan, Developmental Biology of Flowering Plants, Chapters 4 and 6, Springer-Verlag New York, Inc. (2000)). Genes that affect xylem formation in these systems can be identified, isolated, and functionally characterized. The Zinnia elegans mesophyll cell culture system is particularly useful for investigating the molecular basis of xylem formation. Isolated mesophyll cells of Zinnia leaves synchronously trans-differentiate into tracheary elements in the presence of auxin and cytokinin. This trans-differentiation process involves cell commitment, secondary cell wall thickening and lignification and cell autolysis. See, e,g., H. Fukudo and A. Komamine (1980), Plant Physiol. 65: pp. 57-60 and 61-64; D. L. Church (1993), Plant Growth Reg. 12: 179-188 (review). Various aspects of xylogenesis have been studied in this system and several endogenous genes that are involved in the process have been identified.

There is considerable need in the art for a rapid and sensitive in vitro assay to identify genes involved in the regulation of processes involved in xylogenesis which can be used to improve commercially desirable plant traits (e.g., enhanced digestibility of forage crops by animals, increased processability of wood and crops for energy production and pulping, increased mechanical strength of plants and resistance to pests and pathogens and others). Ideally, such an assay would be useful for screening for promoters that are active in vascular tissues and transcriptional regulators that could be used to produce transgenic plants and trees with desirable traits.

### SUMMARY OF THE INVENTION

The present invention provides functional assays for high-throughput screening and identification of genes that affect xylogenesis and their transcriptional regulators. Such genes may be obtained from any type of plant but preferably are derived from forage crops and woody plants, e.g., grasses, grains, legumes, forestry trees and the like. The assay may be used to identify, for example: genes and promoters of genes for transcription factors, co-activators and repressors which function in vascular tissues; genes and promoters of genes involved in xylogenesis and wood formation; genes and promoters of genes involved in cell wall biosynthesis and/or cell wall thickening; genes and promoters of genes involved in the formation of tracheary elements which affect various properties of tracheary elements, such as morphology, architecture and composition of secondary cell walls; and genes and promoters of genes involved in signal transduction events that relate to differentiation and programmed cell death.

In one of its aspects, the invention provides a functional assay for the identification of plant polynucleotide sequences that are active during xylogenesis, comprising transforming Zinnia mesophyll cells with a DNA construct comprising a polynucleotide sequence to be tested for activity, comparing the activity of the test sequence under non-inducing and transdifferentiation-inducing conditions, and identifying sequences that are more active in the cells under inducing conditions than non-inducing conditions.

In one of its embodiments, the assay employs promoter-reporter gene constructs to identify gene promoters or portions thereof which are active during xylogenesis. In other embodiments, the assay employs effector constructs to identify polynucleotide sequences encoding transcriptional regulators of promoters which are active in xylem and xylem-forming tissues and polynucleotide sequences encoding proteins that affect tracheary element formation and processes.

In another aspect, the invention provides a composition for use in identifying transcription factors for promoters of tree genes that are active in xylogenesis comprising Zinnia mesophyll cells transformed with promoters of tree genes that are expressed during xylogenesis.

In yet another aspect, the invention provides a composition for use in identifying promoters of tree genes that are involved in xylogenesis comprising Zinnia mesophyll cells transformed with transcription factors that are active in xylem-forming tissues.

### BRIEF DESCRIPTION OF THE FIGURES

FIG. 1. Temporal induction of E. grandis OMT and Arab-1 xylem-specific promoter activity in transdifferentiating Zinnia elegans cells.

FIG. 2. Specific induction of OMT promoter activity in trans-differentiating Zinnia elegans cells co-transfected with OMT/EGFP and CaMV35S/DsRed 2.

FIG. 3. Schematic diagram of E. grandis OMT promoter deletions.

FIG. 4. Schematic of promoter-reporter gene construct

FIG. 5. Schematic of basic effector DNA construct. The construct comprises a relatively small plasmid backbone that allows replication and selection in an E. coli host (e.g., pUC), a promoter (test or sham), a 3′ untranslated region and an open reading frame encoding a test protein (e.g., a transcription factor).

FIG. 6. Schematic of modified effector DNA construct designed specifically for transcription factors. The construct includes a translationally fused nuclear localization sequence and an activation or repression domain.

FIG. 7. Activation of E. grandis OMT promoter EGX002193HT (534bp) and 306bp, 119bp and 66bp fragments by E. grandis transcription factor EGIA012123HT measured by GUS expression. The fluorescence is represented as arbitrary fluorescence units (FU) per microgram protein per minute.

FIG. 8. Activation of E. grandis OMT promoter EGX002193HT 485bp and 99bp fragments by E. grandis transcription factor EGIA012123HT measured by GUS expression. The fluorescence is represented as arbitrary fluorescence units (FU) per microgram protein per minute.

FIG. 9. Activation of E. grandis OMT promoter EGX002193HT by E. grandis transcription factor measured by EGFP expression relative to the expression of DsRed2 driven by the CaMV 35S promoter.

FIG. 10. Activation of E. grandis arabinogalactan-like 1 promoter EGX015163HT by E. grandis transcription factor EGIA012123HT. The fluorescence is represented as arbitrary fluorescence units (FU) per microgram protein per minute.

### DETAILED DESCRIPTION

The present application relates to a functional assay for high-throughput screening and identification of plant polynucleotide sequences that affect xylogenesis and properties of plants related thereto. The assay involves transforming Zinnia elegans mesophyll cells under non-inducing or transdifferentiation-inducing conditions (hereafter referred to respectively as non-induced or transdifferenting cells) with DNA constructs comprising one or more heterologous polynucleotide sequences to be tested, and comparing the expression of the sequences in the non-induced and transdifferentiating cells. In preferred embodiments, the test polynucleotide sequences are derived from forage crops (e.g., grasses, including switchgrass and ryegrass, legumes, sorghum, maize, other forage crops), forestry trees (e.g., pine and eucalyptus, poplars, sweetgum, spruce, and others) and other woody plants. As the assay requires only that the test polynucleotide sequence have a measureable effect on processes involved in trans-differentiation and/or tracheary element formation, it is contemplated that polynucleotide sequences from other plants may be usefully tested in this assay.

More specifically, the assay is useful for identifying genes that are expressed in vascular tissues (e.g., cambium and xylem) such as genes that encode transcriptional regulators, enzymes and other proteins involved in the formation, architecture and composition of secondary cell walls, e.g., in lignin, cellulose and hemicellulose biosynthesis, in tracheary element formation and properties, in signal transduction, programmed cell death, and the like.

The assay is useful for identifying promoters of genes that are expressed in vascular tissues and their transcriptional regulators. Also, as disclosed herein, the assay may be used in conjunction with well-known methods such as deletion analysis, linker scanning mutation analysis and others to identify active promoter fragments and cis-regulatory elements. Synthetic promoters can be constructed aided by the assay. The assay provides a rapid means for functional testing and annotation of structural homologs of tree genes having identified roles in xylogenesis and related processes.

Definitions

The phrase “Zinnia mesophyll cells” refers to mesophyll cells that are isolated from leaves of Zinnia, preferably Zinnia elegans, and their protoplasts. Exemplary protocols are provided in Examples 1 and 2 below.

The term “heterologous polynucleotide” refers to a polynucleotide that does not naturally occur in the cell into which it is transferred.

A polynucleotide sequence such as a promoter or a protein-encoding sequence which is “active in xylem” or “xylem-specific” refers to a sequence that is activated or expressed selectively or to a greater degree in xylem or in xylem-forming tissue than in other plant tissues.

The phrase “transdifferentiation-inducing conditions” is used interchangeably with “tracheary element-inducing conditions” to refer to incubation conditions which induce Zinnia elegans mesophyll cells to form tracheary elements (TE) in vitro. It should be understood that conditions that differ in certain respects from those described in this application (e.g., hormone composition, hormone levels, media, and time of culturing) can be substituted provided they are capable of inducing trans-differentiation. Cells that are induced to trans-differentiate are referred to as “trans-differentiating cells.

The term “FKH medium” refers to the transdifferentiation-inducing medium described below under General Methods. As described previously in this application and the cited references, the process of “trans-differentiation” involves multiple events and culminates in the formation of tracheary elements.

The term “transforming” refers to the process of introducing recombinant DNA into the plant cell for transient expression assays. Transformation methods that are exemplified here include biolistic particle transformation and PEG-mediated protoplast transfection. However, other transformation protocols that are well-known in the art may be substituted for those described in this application without changing the nature of the invention.

The phase “non-inducing conditions” is used interchangeably with “maintenance conditions”. Cells cultured under non-inducing conditions described in this application undergo expansion but do not trans-differentiate. The term “FK medium” refers to the culture medium described below under General Methods.

The term “promoter” is used here to refer to a 5′ non-coding sequence of a gene, or one or more portions of the 5′ non-coding region, that is active in transcription. A “minimal promoter” is the minimal transcriptional regulatory sequence required to initiate transcription of an operably linked DNA sequence. Cis-regulatory elements are transcriptional regulatory elements in a promoter region that respond to trans-acting factors (e.g., transcription factors, hormones, environmental signals, tissue-specific factors and the like). A “synthetic promoter” refers to a promoter comprised of individual or multiple characterized cis-elements that mediate gene expression when inserted upstream of a minimal promoter.

The term “transcription factor” refers to a protein other than RNA polymerase which interacts with specific cis-regulatory elements and stimulates or represses transcription. Transcription factors generally contain regulatory domains which increase (activate) or decrease (repress) the rate of transcription. A “transcriptional activator” is a protein which acts alone or is complexed with other proteins to activate gene expression. An activator typically has a DNA binding domain and an activation domain. A “co-activator” lacks DNA binding specificity but enhances or represses transcription.

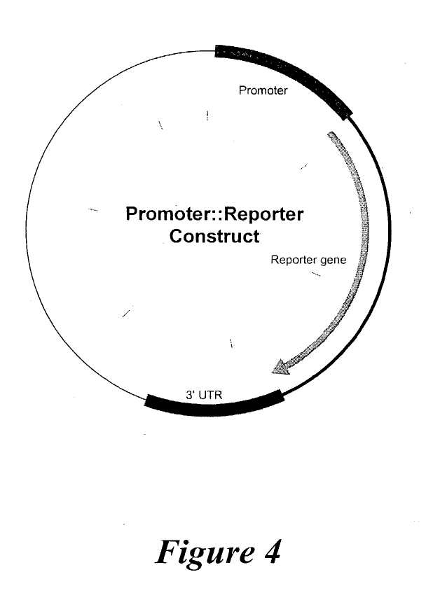

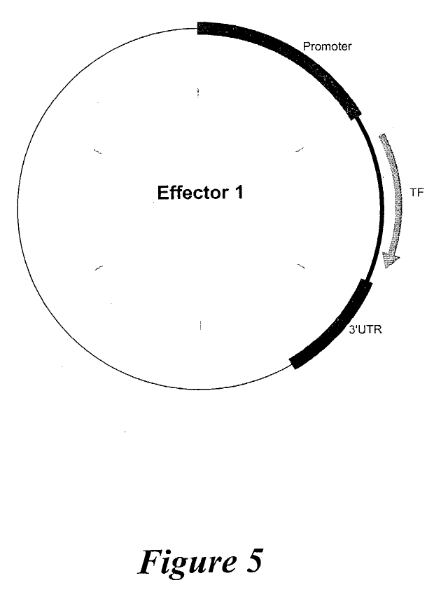

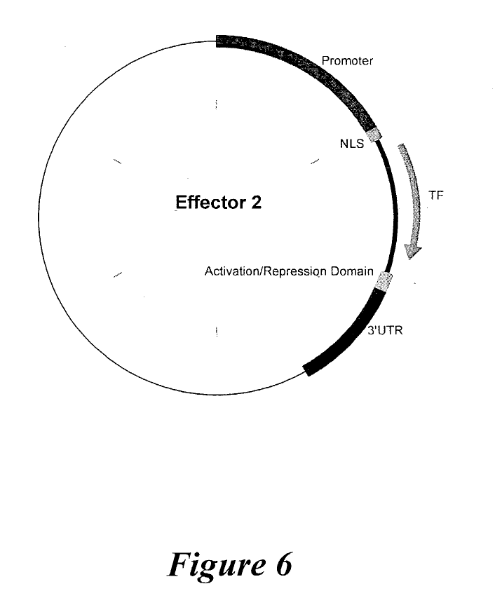

The term “DNA construct” as used herein refers to a recombinant DNA molecule which can be cloned into a vector. The vector may be, for example, a plasmid which is self-replicating in a bacterial cell and contains a selection marker. Exemplary constructs used in embodiments of the assay system described herein are illustrated schematically in FIGS. 4-6 below.

The term “effector DNA construct” is used herein to refer to an expression cassette comprising a coding polynucleotide sequence operably linked to a promoter and termination sequence for controlled expression of the gene in a host cell. The term “operably linked” means that the coding sequence is inserted into the vector in the proper orientation and correct reading frame for translation and its expression is dependent upon interaction of the regulatory sequences with regulatory factors. Techniques for operatively linking the components of the genetic constructs are well known in the art and include the use of synthetic linkers containing one or more restriction endonuclease sites as described, for example, by Sambrook et al., (Molecular cloning: a laboratory manual, CSHL Press: Cold Spring Harbor, N.Y., 1989).

Examples of useful terminators include, without limitation, the Cauliflower mosaic virus (CaMV) 35S terminator, the Agrobacterium tumefaciens nopaline synthase or octopine synthase terminators, the Zea mays zin gene terminator and the Oryza sativa ADP-glucose pyrophosphorylase terminator. Examples of useful promoters which are constitutively active in plants include the CaMV 35S promoter, the nopaline synthase promoter, the octopine synthase promoter, the SuperUbiquitin promoter from Pinus radiata (U.S. Pat. No. 6,380,459) and the Ubi 1 promoter from maize. Exemplary inducible and tissue-specific plant promoters are well known in the art and are described, for example, in WO 02/00894, which is herein incorporated by reference. Other regulatory sequences may be included in the expression cassette (e.g. sequences that are known in the art to enhance translation, to increase nuclear import and the like).

An “effector construct” comprising an open reading frame of a transcription factor may be engineered to include a translationally fused nuclear localization sequence (NLS) and an activation or repression domain to ensure nuclear import of the expressed transcription factor and its ability to activate transcription. For a review on nuclear targeting in plants, see Raikhel, Nev. 1992, Plant Physiol. 100: 1627. See also, Zhao et al., 2001, J. Gen. Virology 82: 1491-1497, for references to nuclear targeting sequences in viruses.

A “sham construct” refers to a negative control construct comprising an open reading frame encoding a test protein which cannot be expressed (e.g., a promoterless construct).

A DNA construct may further include a marker for identifying transformed cells. Suitable markers are well known in the art and include genes that confer resistance to antibiotics, herbicides and other toxins, (see, e.g., Yoder and Goldsbrough, 1994, Bio/Technology 12: 263-267), or reporter genes that encode proteins that are foreign to the host cell and generate fluorescent, chemiluminescent, phosphorescent or luminescent signals upon excitation. See Schrott, M., 1995, In: Gene Transfer to Plants (Potrykus, T. spangenbert, Ed.) Springer Verlag. Berlin, pp. 325-336. Many different reporter genes are known in the art, e.g., firefly luciferase (de Wet et al. 1987, Mol. Cell. Biol. 7: 725-737); bacterial luciferase (Engebrecht and Silverman, 1984, Proc. Natl. Acad. Sci. USA 1: 4154-4158); phycobiliproteins; green fluorescent protein (GFP; T sien R Y, 1998, Ann. Review of Biochemistry 67: 509-544; Haseloff, J. and Amos, B., 1995, Trends Genet. 11: 328-329.). See also, US20020119498A1 and U.S. Pat. No. 6,096,947 which reference GFP mutants and synthetic constructs that encode GFP with enhanced brightness and/or modified spectral characteristics (e.g., enhanced green fluorescent protein, blue fluorescent protein, red fluorescent protein and others). Other reporter genes encode enzymes which produce fluorescent products from fluorescently labeled substrates (e.g., β-galactosidase acting on fluorescein di-β-D-galactopyranoside; β-glucuronidase (GUS) (Jefferson et al., 1987, EMBO J. 6: 3901-3907). Ideally a reporter gene has low background activity, is detectable over autofluorescent plant materials (e.g., chlorophyll and other pigments), is nontoxic to the host cell and does not perturb metabolic processes, the reporter gene product is stable over the experimental period and, for quantitative measurements, is detectable over a large linear range using non-destructive, easily performed and inexpensive procedures.

A “promoter-reporter construct” comprises an open reading frame of a reporter gene operably linked to a promoter (functional or sham) and a termination sequence. It is well known in the art to use multiple reporter genes in cell based assays to increase the reliability of data interpretation and to monitor multiple processes or interactions. Fluorescent reporter genes that encode proteins with emission or excitation spectra that can be simultaneously detected in a single cell using detection devices equipped for multiparameter fluorescence analysis are commercially available. Between four and five fluorescent gene products (e.g., DsRed, EYFP, EGFP, ECFP, EBFP) can be simultaneously monitored in living cells by flow cytometry procedures (Hawley et al, Biotechniques (2001) 30:1028-1034; U.S. Pat. No. 6,096,947). The reporter genes may be provided on the same or separate plasmids. Certain embodiments of the cell-based assay of the present invention involve triple transfection protocols in which reporter, effector and transfection marker plasmids are simultaneously introduced into Zinnia protoplasts.

Description of the Assay

Mesophyll cells are prepared from primary and secondary pair leaves of Zinnia elegans seedlings, then cultured in inducing (FKH) or non-inducing medium (FK) for periods of time spanning the trans-differentiation process (typically, the time period of incubation is overnight to 3 days). Cultured mesophyll cells and freshly prepared mesophyll cells that have not been exposed to culture conditions are transformed with sham DNA constructs or effector DNA constructs. It is useful to include freshly prepared mesophyll cells as a control for potential changes in transgene expression that result from expansion of cells under non-inducing culture conditions. The transfected protoplasts and/or transformed cells are harvested by centrifugation and assayed for viability and transgene expression. To correct for experimental variation that may arise from differences in PEG-mediated transfection of expanding noninduced cells and transdifferentiating cells, which complicates the interpretation of reporter gene expression data, the protoplasts may be cotransfected with a construct comprising a transfection marker under the control of a constitutive promoter (e.g., a transfection marker sequence encoding a fluorescent protein that is detectable in the presence of a different fluorescent reporter gene). Appropriate combinations of commercially available markers and reporter genes can be selected without undue experimentation by those of ordinary skill in the art.

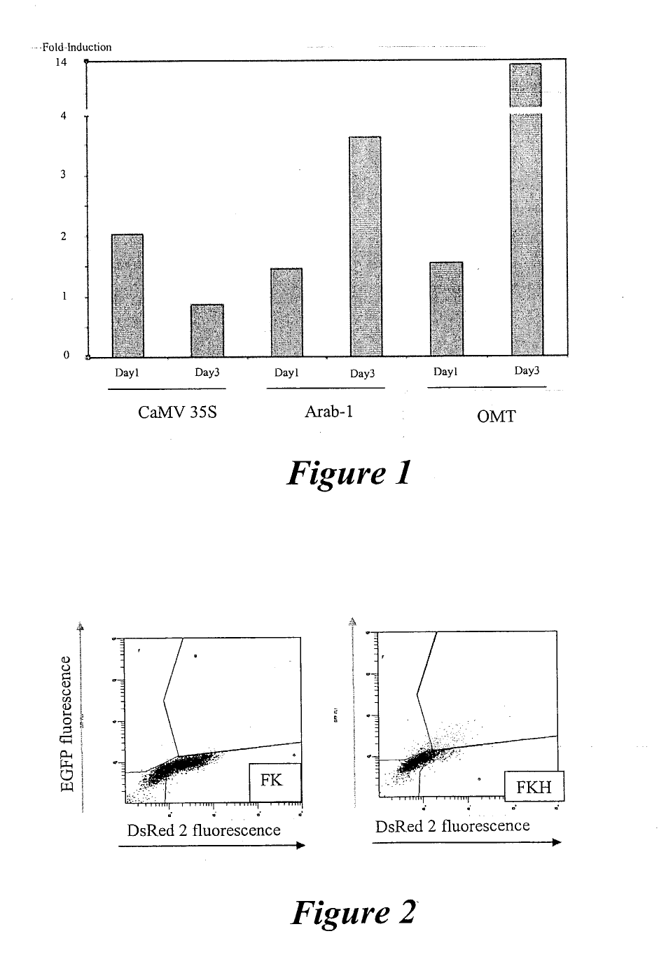

In one of its embodiments, the assay is used to identify promoter sequences that are active in xylem. The identification of promoters depends on the presence of appropriate transcriptional machinery in transdifferentiating Zinnia elegans mesophyll cells. In an initial experiment to determine whether the requisite transcriptional machinery was present, we compared the expression of the GUS reporter gene under the control of Eucalyptus grandis xylem-specific promoters (the OMT promoter and the Arabinogalactin-like 1 promoter) with GUS expression driven by the constitutive CaMV 35S promoter in transdifferentiating Zinnia elegans mesophyll cells. Promoter-reporter gene constructs were transfected into protoplasts prepared from Zinnia elegans mesophyll cells that had been cultured in TE-inducing medium (FKH) for 1 or 3 days, and GUS activity was assayed 24 hours later, as described in Example 4 below. Enhanced activity of both the OMT promoter (14-fold induction) and the Arab-1 promoter (3-4 fold induction) was observed in older (3 day) compared with younger (1 day) transdifferentiating mesophyll cells, whereas activity of the CaMV 35S promoter was not affected by the transdifferentiation process (FIG. 1). FIG. 2 shows a comparison of constitutive CaMV 35S promoter activity and OMT promoter activity in Zinnia elegans cells that were cotransfected with constructs comprising CaMV 35S/DsRed2 and OMT/EGFP and cultured for three days in FK or FKH media. Analysis of the fluorescence cytometry data shows that the red fluorescence levels of the transfected cell population remain unchanged, while the green fluorescence levels are markedly increased under inducing conditions. These experiments demonstrate that the transcription machinery required to activate xylem-specific Eucalyptus gene expression is present in an inducible form in Zinnia elegans cells. Similar experiments have identified other xylogenesis-related promoters from Eucalyptus and Pine (data not shown).

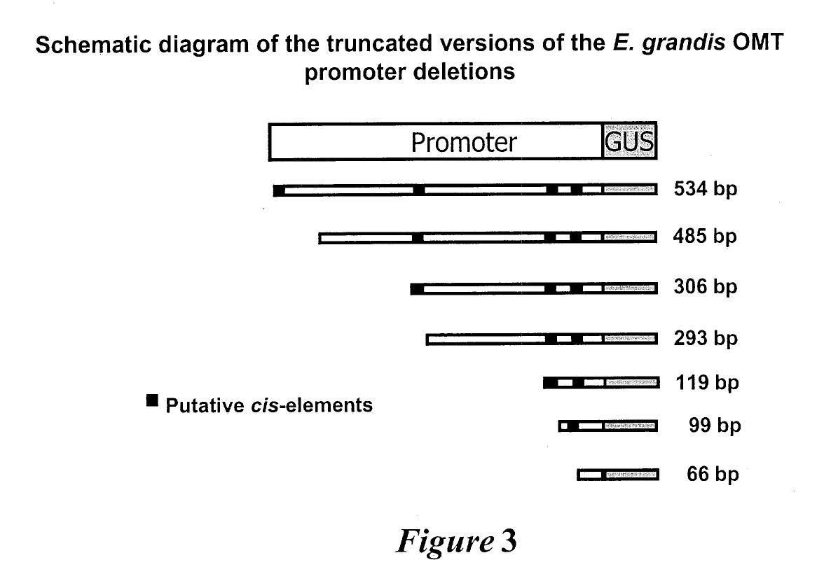

In another of its embodiments, the assay is used to functionally characterize the transcriptional regulatory elements of xylem-specific promoters such as those identified as described above. A series of promoter fragments were designed to dissect the functional cis-elements that reside in the 534 bp OMT promoter, based on the position of putative vascular specific and PAL/AC rich cis-elements in the promoters of other plant genes (GXXXGTTG, Ringli and Keller, 1998, Plant Molecular Biology 37: 977-988; CCATAAACCCC, Kawaoka et al, 2000, Plant Journal 22: 289-301; CCACCTACC, Lacombe et al, 2000, Plant Journal 23: 663). Reporter gene constructs comprising each of the fragments were made and tested for inducibility in the TE-forming assay. A schematic diagram of the truncated versions of the E. grandis 534 bp OMT promoter that were tested is shown in FIG. 3. The 534 bp promoter and fragments of 99 bp, 119 bp, 293 bp, 306 bp, and 485 bp showed enhanced activity in TE-forming cells. There is evidence that an enhancer element is localized between 293 and 119 bp. A minor reduction in reporter gene activity was found when the PAL/AC rich box located in the sequence between the 306 bp fragment and the 293 bp fragment was deleted. Reporter gene constructs comprising the 534bp promoter, and 119 bp, 306 bp and 485 bp promoter fragments were tested in transgenic tobacco plants. All of these fragments were preferentially expressed in vascular tissue.

The effects of cytokinin and auxin on promoter activity were tested in Zinnia elegans cells transfected with the 306 bp and 119 bp promoter constructs and cultured in FK medium. Auxin was sufficient to induce promoter activity (data not shown here). Hormone responsiveness is confined to a 33 bp region containing an AC rich/L box-type element (data not shown here).

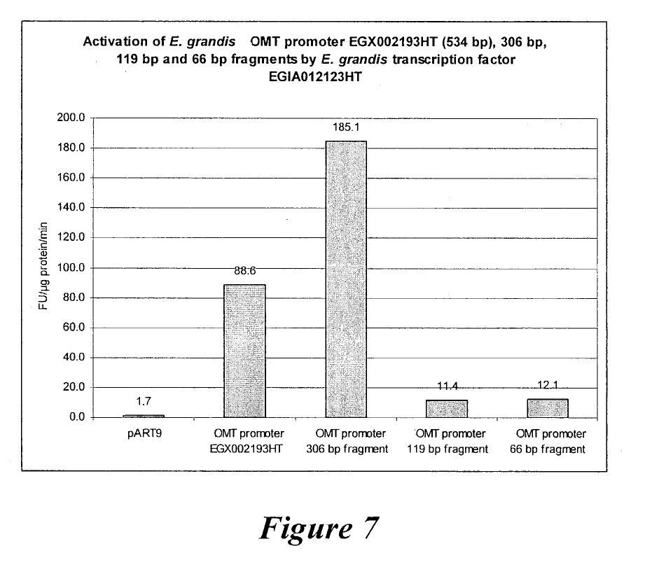

In another of its embodiments, the assay is used to identify genes that encode transcriptional activators and repressors of xylem-specific promoters (see Examples 6-8 and FIGS. 7-10 below). In this embodiment, Zinnia elegans cells are cotransfected with an effector construct comprising a test transcriptional regulator fused to a constitutive promoter and a promoter-reporter gene construct, where the promoter or a promoter fragment is known to be active in xylem or has been identified as such in the assay. As mentioned previously, at least three different constructs, including a transfection marker, may be simultaneously transfected into the cells and analyzed with appropriate reporter genes and detection devices. An analysis of changes in reporter gene signal under inducing and non-inducing conditions with and without the expressed transcriptional regulator will indicate whether the promoter or promoter fragment is activated or repressed by the test transcriptional regulator. The results suggest that the inventive assay is useful for identifying transcription factors that act on promoters of genes which are normally confined to and expressed in xylem and xylem-forming tissues. For example, various transcription factors from Pine and Eucalyptus have been identified with the assay.

In yet another embodiment, the assay may be used to identify genes that affect various properties of the tracheary element morphology, architecture, and composition of the secondary cell walls. Genes that affect crystalline cellulose content, protein and polysaccharide composition, the content and composition of lignin and monolignols, localization of cell wall components, diameter and shape of tracheids and thickness of cell wall layers, and other features of xylogenesis in the Zinnia transdifferentiation system can be analyzed by standard methods, which include but are not limited to TEM, SEM, light microscopy, laser scanning confocal fluorescence microscopy), FACS, HPLC, GC-MS, FTIR, tissue print hybridization and other molecular biological techniques, and other well-known chemical/biochemical/immunochemical methods. In still yet another embodiment, the assay may be used to identify genes that affect signal transduction events involved in differentiation and programmed cell death The analysis of these events includes examination of the culture media of trans-differentiating cells for secreted diffusible mediators of cell interactions.

In yet another embodiment, the assay may be used to assess the effects of hormones and other compounds (e.g., cellulose synthesis inhibitors such as isoxaben and dichlobenil) on the trans-differentiation process and the expression of plant genes that are active in xylogenesis.

The throughput of the assay can be increased by high throughput cloning of genes to be tested and by automating certain aspects of the assay. In addition, the assay can be multiplexed by increasing the number of inputs (e.g., plasmid constructs simultaneously transfected) and/or increasing the number of outputs that are measured from a single assay. Examples include assaying co-effector or repressor plasmids together with transcription factors and measuring protein expression, cell cycle status and cell wall composition using multiple output readers.

### EXAMPLES

The following non-limiting methods and examples are provided to illustrate the practice of the invention.

### Example 1

### Isolation and Culture of Zinnia elegans Mesophyll Cells in Tracheary Element (TE) Inducing (FKH) and Non-Inducing (FK) Medium

Primary and secondary pair leaves from the Zinnia seedlings were harvested from 8 punnets. Leaves were sterilized in 500 mL of 0.175% sodium hypochlorite solution for 10 minutes. Leaves were then rinsed twice in 500 mL of sterile water. Using 20-30 leaves at a time, leaves were ground in mortar and pestle and 25-30 mL of FK medium. Cells were filtered through the 40 μm nylon mesh. A total of 90 mL of mesophyll cells were obtained in this fashion. Cells were pelleted by centrifuging at 200×g for 2 minutes at 20° C. The pellet was washed once more using equal volume of FK medium. Then the pellet was split in to two equal halves and one half was washed in 45 mL of FK medium and the other in 45 mL of FKH medium. The pellets were re-suspended in 60 mL of FK medium and 60 mL of FKH medium, respectively. They were cultured in the dark in two 6-well plates on the rotary shaker set at 120 rpm.

### Example 2

### Isolation of Zinnia elegans Protoplasts from Leaves or Mesophyll Cells Cultured Overnight to Three Days in FK Medium and FKH Medium

Sterile Zinnia elegans primary leaves (6-8 in number) were cut in slivers of 1 mm and placed in 15 mL of cell wall digesting enzyme mix (1% Cellulase Onozuka R-10 and 0.2% pectolyase Y23 in Protoplast isolation buffer). Mesophyll cells cultured in FK medium (40 mL) or FKH medium (40 mL) were pelleted by centrifuging at 200×g for 2 minutes at 20° C. Each pellet was re-suspended in 20 mL of sterile Protoplast isolation buffer containing 200 mg Cellulase Onozuka R-10 and 40 mg Pectolyase Y23. The protoplasts were isolated by incubating the cell suspensions in CellStar culture plates for 2-4 hours on a rotary shaker set at ˜70 rpm at 23° C. Protoplasts were pelleted by centrifuging the contents of the plates at 200×g for 2 minutes. Each of the pellets was re-suspended in 20 mL of 24% sucrose solution.

### Example 3

### Transfection of Zinnia elegans Protoplasts

Zinnia protoplasts in 24% sucrose solution were overlaid with 1 mL of W5 solution and centrifuged at 70×g for 10 minutes at 20° C. with brakes off. Floating protoplasts were harvested and resuspended in 10 mL of W5 solution. Protoplasts were pelleted by centrifuging at 70×g for 10 minutes at 20° C. Protoplasts were resuspended in MaMg medium (density=˜5×10⁶ protoplasts/mL) and aliquoted into individual 15 mL tubes (300 μL: 1.5×10⁶ protoplasts). 5 μg DNA (of each construct) and 50 μg Salmon Testes DNA was added to the protoplast suspension, mixed and incubated for 5 minutes at 20° C. 300 μL 40% PEG solution was added to each aliquot of protoplasts, mixed and incubated for 20 minutes at 20° C. 5 mL of K3/0.4M sucrose was added to each aliquot of leaf-derived transfected protoplasts or transfected protoplasts from mesophyll cells cultured in FK medium and mixed. Similarly, 5 mL of K3/0.4M sucrose+0.1 ppm NAA+0.2 ppm BA was added to each aliquot of transfected protoplasts from mesophyll cells cultured in FKH medium and mixed. The transfected protoplast suspensions were incubated overnight at 23° C. in the dark.

### Example 4

### Harvesting of Transfected Zinnia elegans Protoplasts and Reporter Gene Analysis

Transfected Zinnia protoplast suspensions prepared as described above were individually harvested by adding 9.5 mL of W5 solution, mixing the contents of each tube and centrifuging at 70×g for 10 minutes at 20° C. The bulk of the supernatant was removed by decanting and the protoplasts volume was brought up to 900 μL. From this, 300 μL of protoplasts were aliquoted into 5 mL polystyrene round-bottom tubes, re-suspended in a volume of 500 μL W5 medium and set aside for analysis of fluorescent reporter gene expression and cell viability. The protoplasts and the remaining solution were transferred to individual microtubes and pelleted by centrifugation at 420×g for 2 minutes at 20° C. The protoplast pellet was assayed for GUS reporter gene expression. as described by Jefferson, R. A., 1987, Plant Mol Biol. Rep. 5, 387. GUS (MUG) assays were performed using a Wallac (Turku, Finland) Victor² 1420 Multilabel Counter. Umbelliferone was detected using a 355 nm excitation filter and a 460 nm emission filter for 1 second.

### Example 5

### Biolistic-Mediated Transformation of Zinnia elegans

A. Plasmid Coating of Gold Particles.

Materials: DNA constructs (50 μg: 1 μg/μL), 0.6 μm gold particles (Bio-Rad), 100 μL of 50 mM Spermidine (Sigma), Gold-coat Tefzel™ tubing (Bio-Rad).

100 μL of spermidine was added to 6.3 mg of 0.6 μm gold particles. The mixture was vortexed and sonicated for 3-5 seconds. 50 μL of DNA was added to the gold particles, vortexed and 100 μL of 1 M CaCl₂ was added drop-wise while mixing. The mixture was allowed to precipitate at room temperature for 10 minutes, then microfuged for 15 seconds to pellet the gold particles. The supernatant was removed and discarded and the pellet was washed with 1 mL of fresh 100% ethanol, microfuged for 5 sec and the supernatant was discarded. This procedure was repeated for a total of three times. The pellet was resuspended in a tube in 200 μL of PVP (MW 360,000) in ethanol solution (0.05 mg/ml) and the volume was adjusted to 3.5 mL with the PVP ethanol solution.

The DNA/gold particles were coated to the Tefzel tubing as per manufacturer's recommendation and the prepared bullets stored at 4° C. in a bottle containing a desiccant.

B. Bombardment of Cells in Culture.

The Helios gene gun was loaded as per manufacturer's recommendation. The helium regulator was adjusted to about 150-200 psi pressure for delivering the DNA/gold to the target tissue.

Isolated Zinnia mesophyll cells in culture medium were pelleted and 60 μL pellets were used for bombardment. Cells were bombarded for a total of three times. Post-bombardment, the cells were incubated in 3 mL of FK or FKH medium.

Samples were analyzed after an overnight incubation on a rotary shaker set at ˜140 rpm in dark at 23° C.

### Example 6

### Activation of E. grandis OMT Promoter by E. grandis Transcription Factor EGIA012123HT Measured by GUS Activity

Following the protocols described above, the E. grandis transcription factor EGIA012123HT was tested for its ability to activate the E. grandis OMT promoter EGX002193HT.

Specifically, Z. elegans protoplasts were co-transfected with two of three disparate constructs. Test protoplasts were transfected with the effector construct, pFOR263. Constructs of the pFOR series are based on the primary cloning vector pART7, which has an expression cartridge comprised of the CaMV 35S promoter, a multiple cloning site and the transcriptional termination region of the octopine synthase gene (Gleave, Plant Mol. Biol. 20:1203-1207, 1992). The vector pFOR263 contains the E. grandis MYB transcription factor, EGIA012123HT, in its multiple cloning site. The protoplasts were also transfected with a second plasmid containing the GUS gene driven by the E. grandis COMT promoter EGX0002193HT or deletion fragments of the promoter.

Control protoplasts were transfected with a plasmid vector, pART9, a modified version of pART7, containing the GUS gene in its multiple cloning site but with the CaMV 35S promoter removed from the expression cartridge. Accordingly, pART9 is a sham construct which does not express any gene and is used as a control because of its similarity in length and composition to pFOR vectors. The control protoplasts were also transfected with the second plasmid containing the GUS gene driven by the E. grandis OMT promoter EGX0002193HT.

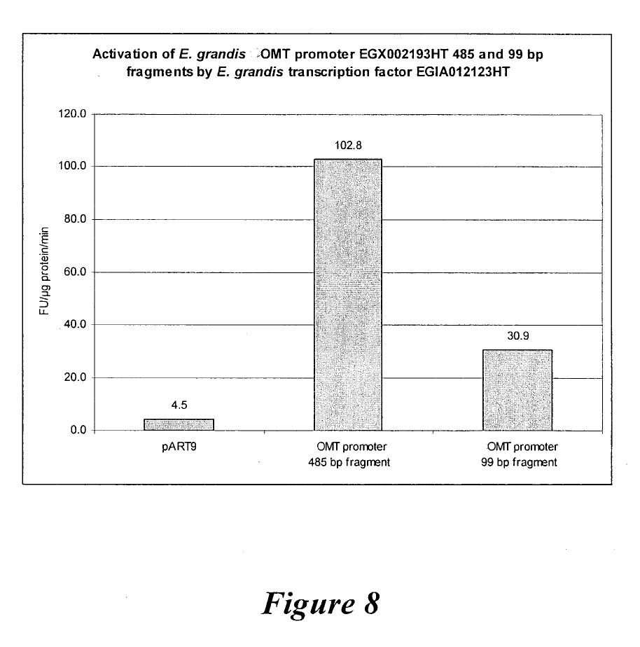

The results of two separate experiments are shown in FIGS. 7 and 8. Both graphs show strong activation of the E. grandis OMT promotor and promoter fragments by the E. grandis transcription factor EGIA012123HT compared to the controls.

### Example 7

### Activation of E. grandis Arabinogalactan-Like 1 Promoter (Arab-1) by E. grandis Transcription Factor EGIA012123HT Measured by GUS Activity

Following the protocols described above, the activation of the E. grandis arabinogalactan-like 1 promoter, Arab-1 by E. grandis transcription factor EGIA012123HT was analyzed.

In particular, Z. elegans protoplasts were co-transfected with two of three disparate constructs. Test protoplasts were transfected with the effector construct, pFOR263, which contains the E. grandis MYB transcription factor, EGIA012123HT. The protoplasts were also transfected with a second plasmid containing the GUS gene driven by the E. grandis Arab-1 promoter. Control protoplasts were transfected with the plasmid vector, pART9, and a second plasmid containing the GUS gene driven by the E. grandis Arab-1 promoter.

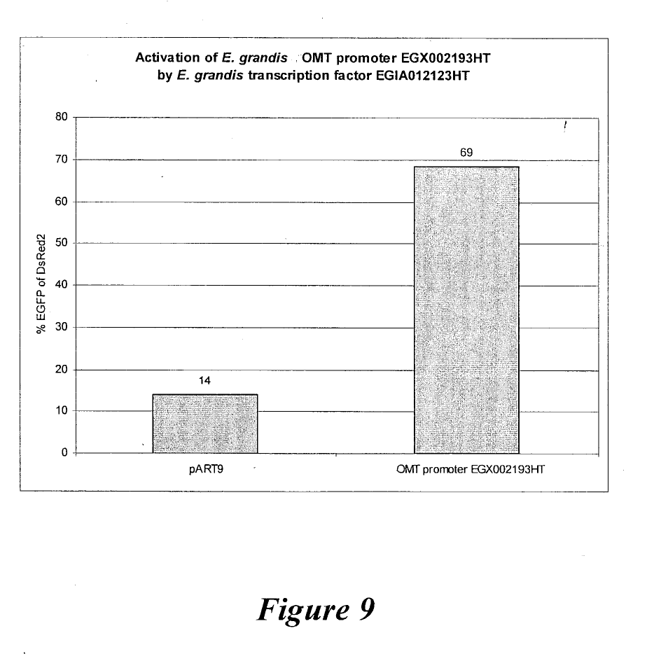

The results in FIG. 9 show that the E. grandis transcription factor EGIA012123HT-containing protoplasts display a significantly stronger activation of the E. grandis Arab-1 promoter than the control protoplasts.

### Example 8

### Activation of the E. grandis OMT 534bp Promoter by E. grandis Transcription Factor EGIA012123HT

Following the protocols described above, the activation of the E. grandis OMT 534 bp promoter by the E. grandis transcription factor EGIA012123HT was tested.

Z. elegans protoplasts were transfected with three of four disparate constructs. Test protoplasts were transfected with pFOR263 containing the E. grandis MYB transcription factor, EGIA012123HT, a second vector containing the DsRed2 reporter gene under the control of a CaMV 35S promoter and a further plasmid containing the E. grandis OMT 534 bp promoter, driving the EGFP reporter gene. Control protoplasts were transfected with the promoter-less pART9, a second vector containing the DsRed2 reporter gene under the control of a CaMV 35S promoter and a further plasmid containing the E. grandis OMT 534 bp promoter, driving the EGFP reporter gene.

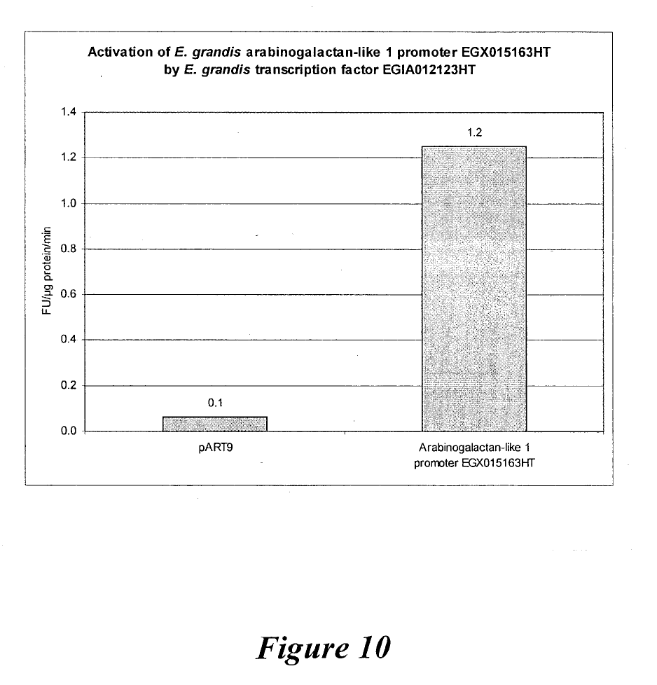

The results in FIG. 10 show the percentage of EGFP fluorescence in relation to DsRed2 fluorescence, i.e. the activity of the E. grandis OMT 534 bp promoter in relation to the activity of the constitutively expressed CaMV 35S promoter. The activity of the E. grandis OMT 534 bp promoter fragment is five times higher in protoplasts containing the E. grandis transcription factor EGIA012123HT than in those containing only the sham construct.

General Methods

| Media Preparation | | |
|---|---|---|
| Fukuda and Komamine Medium Stocks | mg | |
| | Stock A (10x) | |
| KNO₃ | 20,200 | |
| NH₄Cl | 540 | |
| MgSO₄.7H₂O | 2,470 | |
| CaCl₂.2H₂O | 1,470 | |
| KH₂PO₄ | 680 | |
| Milli-Q water | up to | |
| | 1,000 | mL |
| Store at room temperature. | | |
| | Stock B (400x) | |
| MnSO₄.4H₂O | 2,500 | |
| H₃BO₃ | 1,000 | |
| ZnSO₄.7H₂O | 1,000 | |
| Na₂MoO₄.2H₂O | 25 | |
| CuSO₄.5H₂O | 2.5 | |
| Milli-Q water | up to | |
| | 250 | mL |
| Store at room temperature. | | |
| | Stock C (400x) | |
| Na₂EDTA | 3,700 | |
| FeSO₄.7H₂O | 2,800 | |
| Milli-Q water | up to | |
| | 250 | mL |
| Dissolved by stirring for several hours while heating | | |
| at 100° C. | | |
| Filter sterilize and store at 4° C. | | |
| | Stock D (400x) | |
| Glycine | 200 | |
| myo-Inositol | 10,000 | |
| Nicotinic acid | 500 | |
| Pyridoxine Hydrochloride | 50 | |
| Thiamine Hydrochloride | 5 | |
| Milli-Q water | up to | |
| | 250 | mL |
| Filter sterilize and store at 4° C. | | |
| | Stock E (400x) | |
| Folic acid | 50 | |
| Milli-Q water | up to | |
| | 250 | mL |
| Dissolved by adding a small amount of KOH. | | |
| Filter sterilize and store at 4° C. | | |
| | FK medium | |
| For 1 litre: | | |
| Stock A | 100.0 | mL |
| Stock B | 2.5 | mL |
| Stock C | 2.5 | mL |
| Stock D | 2.5 | mL |
| Stock E | 2.5 | mL |
| Sucrose | 10,000 | mg |
| d-(−) Mannitol | 36,000 | mg |
| Milli-Q water | up to | |
| | 1,000 | mL |
| pH | 5.5 | |
| Filter sterilize and store at room temperature. | | |
| | FKH medium | |
| For 1 litre: | | |
| Stock A | 100.0 | mL |
| Stock B | 2.5 | mL |
| Stock C | 2.5 | mL |
| Stock D | 2.5 | mL |
| Stock E | 2.5 | mL |
| Sucrose | 10,000 | mg |
| d-(−) Mannitol | 36,000 | mg |
| 1-Naphthaleneacetic acid (NAA) | 0.1 | mg |
| | (0.1 | ppm) |
| 6-Benzyladenine (BA) | 0.2 | mg |
| | (0.2 | ppm) |
| Milli-Q water | up to | |
| | 1,000 | mL |
| pH | 5.5 | |
| Filter sterilize and store at room temperature. | | |
| W5 medium: | | |
| 150 mM NaCl | | |
| 125 mM CaCl₂.2H₂O | | |
| 5 mM KCl | | |
| 5 mM sucrose | | |
| pH 5.6-6 | | |
| MaMg medium | | |
| 450 mM mannitol | | |
| 15 mM MgCl₂ | | |
| 0.1% MES | | |
| pH 5.6 | | |
| 40% PEG solution | | |
| 40% PEG 3340 | | |
| 100 mM Ca(NO3)2.4H2O | | |
| 0.45 M mannitol | | |
| pH 9.0 | | |
| K3, 0.4 M sucrose | | |
| Murashige and Skoog Plant Salt Base | 4.3 | g |
| Additions: | | |
| Myo-inositol | 100 | mg/L |
| Xylose | 250 | mg/L |
| Thiamin-HCl | 10 | mg/L |
| Nicotinic acid | 1 | mg/L |
| Pyridoxin-HCl | 1 | mg/L |
| NAA | 1 | mg/L |
| Kinetin | 0.2 | mg/L |
| sucrose | 137 | g |
| pH 5.6 adjust with KOH | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

All of the publications, patent applications and patents cited in this application are herein incorporated by reference in their entirety to the same extent as if each individual publication, patent application or patent was specifically and individually indicated to be incorporated by reference in its entirety.

What is claimed is:

1. A functional assay for the identification of plant polynucleotide sequences that are active during xylogenesis, comprising: transforming Zinnia mesophyll cells with a DNA construct comprising a polynucleotide sequence to be tested for activity, comparing the activity of the test sequence under non-inducing and transdifferentiation-inducing conditions, and identifying sequences that are more active in the cells under inducing conditions than non-inducing conditions.

2. The assay according to claim 1, wherein said DNA construct is a promoter-reporter construct comprising a promoter or portion of a promoter to be tested for activity.

3. The assay according to claim 1, wherein said DNA construct is an effector construct comprising a polynucleotide sequence encoding a putative transcription factor.

4. The assay according to claim 1, wherein said DNA construct is an effector construct comprising a polynucleotide sequence encoding a protein that affects tracheary element formation or properties.

5. The assay according to claim 1, further comprising transforming Zinnia elegans mesophyll cells with a promoter-reporter gene construct comprising a constitutive promoter.

6. The assay according to claim 1, wherein comparing the activity of the sequence involves measuring reporter gene signals or protein expression levels in noninduced and transdifferentiating cells.

7. The assay according to claim 1, wherein said test polynucleotide sequence is derived from the group of plants consisting of forage crops, woody plants and forestry trees.

8. The assay according to claim 7, wherein said test polynucleotide sequence is derived from eucalptus or pine trees.

9. An assay for identifying tree genes that affect wood properties comprising:
(a) transforming Zinnia mesophyll cells cultured under tracheary element-inducing conditions with a DNA construct comprising a xylem-specific promoter operably linked to an open reading frame of a test polynucleotide sequence derived from a tree gene; and
(b) detecting the effect of the expression of the test polynucleotide sequence on the morphology, architecture and/or composition of the secondary cell walls of the tracheary elements which are formed.

10. An assay for identifying a transcriptional regulator of a xylem-specific promoter comprising cotransforming noninduced or transdifferentiating Zinnia mesophyll cells with a promoter-reporter gene construct comprising said xylem-specific promoter and an effector construct comprising a constitutive promoter operably linked to a putative transcriptional regulator sequence and determining the effect of overexpressing the transcriptional regulator on the reporter gene expression.

11. An assay for identifying a transcriptional repressor of a xylem-specific promoter comprising cotransforming non-induced or transdifferentiating Zinnia mesophyll cells with a promoter-reporter gene construct comprising said xylem-specific promoter and an effector construct comprising a constitutive promoter operably linked to a putative transcriptional regulator sequence and identifying a transcriptional regulator which when overexpressed reduces the expression of the reporter gene controlled by the xylem-specific promoter.

12. A composition for use in a cell-based assay for identifying transcription factors for promoters of tree genes that are active in xylogenesis, comprising Zinnia mesophyll cells transformed with promoters of tree genes that are expressed during xylogenesis.

13. A composition for use in a cell-based assay for identifying tree gene promoters that are putatively involved in xylogenesis, said composition comprising Zinnia mesophyll cells transformed with transcription factors that are active in xylem-forming tissues.
